(19) United States
(12) Patent Application Publication
(10) Pub. No.: US 2010/0286662 A1
(43) Pub. Date: Nov. 11, 2010

(54) CATHETER AID

(75) Inventor: Harold Lapworth, North Balgowlah (AU)

Correspondence Address: SNELL & WILMER L.L.P. (Main), 400 EAST VAN BUREN, ONE ARIZONA CENTER, PHOENIX, AZ, 85004-2202, US

(73) Assignee: LA RONDE PTY LTD., BOWRAL (AU)

(21) Appl. No.: 12/600,306

(22) PCT Filed: May 13, 2008
(86) PCT No.: PCT/AU08/00671

§ 371 Date: Jul. 7, 2010

(30) Foreign Application Priority Data
May 17, 2007 (AU) 2007902619

(51) Int. Cl. A61M 25/00

(52) U.S. Cl. 604/528

(57) ABSTRACT

A device for the removal and insertion of a supra-pubic catheter. The device has a body comprising a contact surface for contacting with a patient's skin, an opposed pressing surface adapted to have pressure applied thereon by a user and a slot extending from a side edge of the body towards the centre thereof, the slot being adapted to receive the tube of the catheter therein, such that when the catheter is inserted in a patient, the tube of the catheter is substantially perpendicular to the patient's abdomen. The contact surface includes a raised portion adapted to compress the patient's skin when said pressure is applied to the pressing surface, which eases the pain and tissue trauma experienced during insertion and removal of the supra-pubic catheter.

### TECHNICAL FIELD

This invention relates to improvements in the method of removing and inserting catheters. The present invention also relates to devices to assist in the process of removing and inserting catheters, the devices alleviating the pain associated with insertion and removal of catheters. Particularly, the present invention relates to a device that assists in the removal and insertion of supra-pubic catheters and a method of using the device in the removal and insertion of supra-pubic catheters.

### BACKGROUND ART

A catheter is a flexible tube made of latex, silicone, or teflon that can be inserted into the body creating a channel for the passage of fluid or the entry of a medical device. There are many specialized catheter designs. The three major types of catheters are coronary, renal, and infusion. This invention is particularly concerned with renal catheters, and particularly supra-pubic renal catheters, which are renal catheters that are inserted into a patient's bladder directly through the patient's abdominal wall.

The best-known renal catheters are Foley catheters, which are equipped with an inflatable balloon and are used for urine incontinence, dying patients, and bladder drainage following surgery or an incapacitating injury or illness. The Foley catheter is relatively easy to use and is used throughout the world in hospitals, nursing homes, healthcare facilities and home-care settings. These catheters are usually left in place in the body but need to be replaced periodically. Typically, these catheters are replaced on a monthly basis by trained medical personnel. Commonly, when these catheters are removed and a new catheter is inserted, the patient experiences excruciating pain, often there is also associated attendant bleeding, which in some cases may last up to several days and ultimately result in hematuria (blood in the urine). Infections and hospitalisation are common experiences when removing and inserting supra-pubic catheters in a conventional manner.

It is thought that over the time that the supra-pubic catheter is inserted in the patient, the patient's sub-cutaneous tissue begins to adhere to the shaft of the catheter and when the catheter is removed in a conventional way, the sub-cutaneous tissue follows the catheter in its upward movement through the catheter insertion point in the patient's abdomen. This movement of the sub-cutaneous tissue commonly results in excruciating pain for the patient and more often than not, attendant bleeding. The taking of analgesics or other forms of pain relief by the patient prior to or during the procedure of removing and reinserting the catheter has little or no effect on reducing the pain experienced by the patient during the procedure. The pain experienced by the patient leads to anxiety and fear surrounding the procedure, which decreases the quality of life and in some cases results in depression in the patient who may require the administration of anti-anxiety and/or anti-depressant drugs due to the ongoing trauma experienced during the regular catheter replacement procedures. In turn, the negative experience of the patient leads to increased stress levels for the medical clinicians carrying out the procedure.

It is also common for the patient's abdominal muscles to involuntarily go into spasm in response to the supra-pubic catheter being removed. This causes the size of the insertion point or hole in the patient's abdomen being reduced as well as changing the alignment of the hole, thereby making it necessary to apply considerable force to insert the new catheter. In some cases it is not possible to insert a new catheter because the muscle spasms result in complete closure and realignment of the insertion hole. Failure to insert the new catheter within a limited period of time results in hospitalisation of the patient. Therefore, in some cases, hospitalisation is required because the clinical practitioner cannot insert the new catheter in time. This increases the cost to the healthcare system for hospital visits by patients for complications arising from what should be a relatively minor procedure. With a generally aging world population, particularly in developed nations, and with most developed countries healthcare systems struggling to cope with the increased demand for available hospital beds, there is a need to reduce the number of patients that are hospitalised each year for relatively minor procedures that can be better dealt with outside of a hospital environment. At present, in Australia alone there are some twenty to fifty thousand people who have supra-pubic renal catheters installed. It is estimated that the present cost to the Australian health care system because of catheter related illness is somewhere in the vicinity of A$25,000 per person per annum (for the population over the age of 70 years). The bulk of this cost is incurred because of hospitalisation that occurs when infection occurs because of the catheter.

The management of pain during needle insertion has previously been proposed. For example, United States patent application no. US 2006/0270998 discloses a syringe having a pressure ring around the needle point to compress the skin and ease the pain of injection. Similarly, United States patent no. U.S. Pat. No. 6,752,817 discloses a collar to fit around a lancing device to widen the wound for collecting blood. Also, PCT International patent application publication no. WO 2007/015247 discloses a vacuum technique for compressing the skin at the insertion point.

However, none of these previously known patent documents address the problems associated with catheters, and particularly supra-pubic renal catheters that remain in the body and need to be periodically replaced.

It would be desirable to provide a device that ameliorates or overcomes one or more known disadvantages of existing techniques or that may provide a useful alternative to them. It is therefore an object of the present invention to provide a device that assists in ameliorating the pain experienced by a patient associated with insertion and removal of catheters.

### DISCLOSURE OF INVENTION

According to the present invention there is provided a device for the removal and insertion of a supra-pubic catheter in a patient. The device has a body that comprises a contact surface for contacting with the patient's skin; an opposed pressing surface adapted to have pressure applied thereon by a user; and a slot extending from a side edge of the body towards the centre thereof, the slot being adapted to receive an end of said catheter therein. The contact surface includes a raised portion adapted to compress the patient's skin when said pressure is applied to the pressing surface. Preferably, the raised portion is at least partially convex and the slot is adapted to receive the tube of the catheter therein, such that when the catheter is inserted in a patient, the tube of the catheter is substantially perpendicular to the patient's abdomen

Preferably, in use, when removing and inserting the supra-pubic catheter in a patient, the raised portion is adjacent to the catheter entry point into the patient's abdomen. The device is placed around the catheter so that the catheter abuts an end portion of the slot at or near the centre of the body. When removing and inserting the supra-pubic catheter in a patient, a user applies the pressure to the pressing face and the raised portion depresses the region of the patient's abdomen around the entry point of the catheter thereby widening and realigning the entry point of the catheter in the patient's abdomen. This eases the pain and tissue trauma experienced during removal and insertion of the catheter.

Preferably, the pressing surface includes two pressure zones, which are adapted to have said pressure applied thereon, the pressure zones locate the pressure application evenly about the entry point of the catheter in the patient's abdomen. The pressure zones are preferably marked with distinctive indicia to indicate to the user where to apply the pressure on the pressing surface. In one embodiment, the pressure zones are finger depressions. In another embodiment, the pressure zones are raised finger locating pads.

The device is manufactured from surgical grade material, which is preferably a surgical grade synthetic polymer. Therefore, the device is a sterile, single use “throw-away” item.

According to a further embodiment of the present invention there is provided a method of removing and inserting a supra-pubic catheter in a patient having the steps of locating a device adjacent to the entry point of the catheter in the patients abdomen to fit around the catheter; applying pressure onto the device to depress the region of the patient's abdomen around the entry point of the catheter thereby widening and realigning the entry point of the catheter in the patient's abdomen; whilst the pressure is being continually applied onto the device, removing the catheter from the patient and subsequently inserting a new catheter into the patient.

### BRIEF DESCRIPTION OF DRAWINGS

Preferred embodiments of the present invention are illustrated in the drawings, by way of example only, in which:

FIG. 1 is a perspective view of a first embodiment of the device to assist in the removal and insertion of supra-pubic catheters according to the present invention;

FIG. 2 is a plan view of the device shown in FIG. 1;

FIG. 3 is a cross-sectional view of the device shown in FIG. 1 along the line A-A of FIG. 2;

FIG. 4 is a front end view of the device shown in FIG. 1;

FIG. 5 is an enlarged front end view of the device shown in FIG. 1 of the portion B shown in FIG. 4;

FIG. 6 is an upper perspective view of a second embodiment of the device to assist in the removal and insertion of supra-pubic catheters according to the present invention;

FIG. 7 is a bottom perspective view of the embodiment shown in FIG. 6;

FIG. 8 is an upper perspective view of a second embodiment of the device to assist in the removal and insertion of supra-pubic catheters according to the present invention;

FIG. 9 is a top plan view of the device shown in FIG. 8; and

FIG. 10 is front perspective view of the device shown in FIG. 8.

### BEST MODE FOR CARRYING OUT THE INVENTION

This invention relates to improvements in the method of removing and inserting catheters. The present invention also relates to a device to assist in the process of removing and inserting catheters, the device alleviating the pain associated with inserting and removing catheters. Particularly, the present invention relates to a device that assists in the removal and insertion of supra-pubic catheters and a method of using the device in the removal and insertion of supra-pubic catheters.

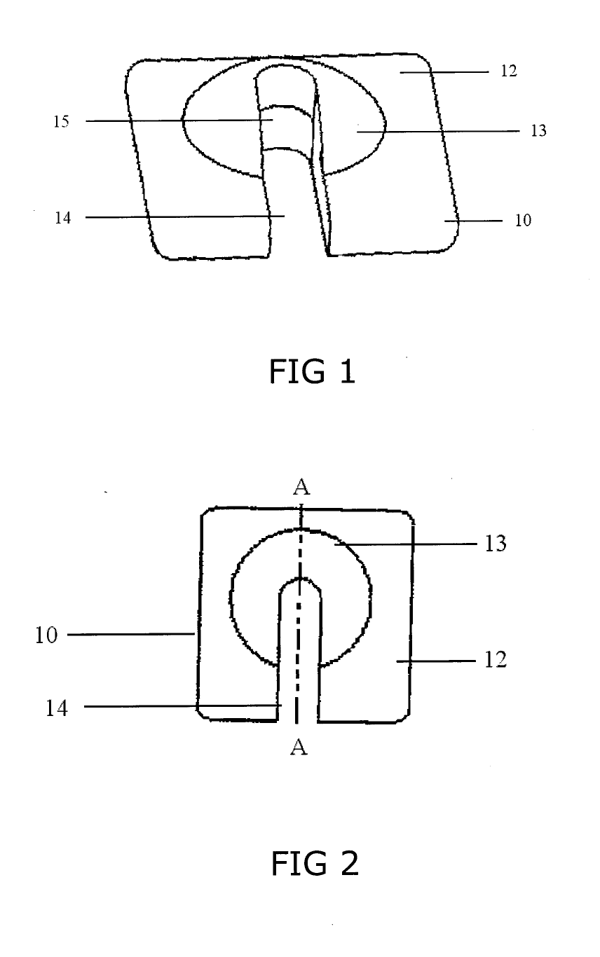

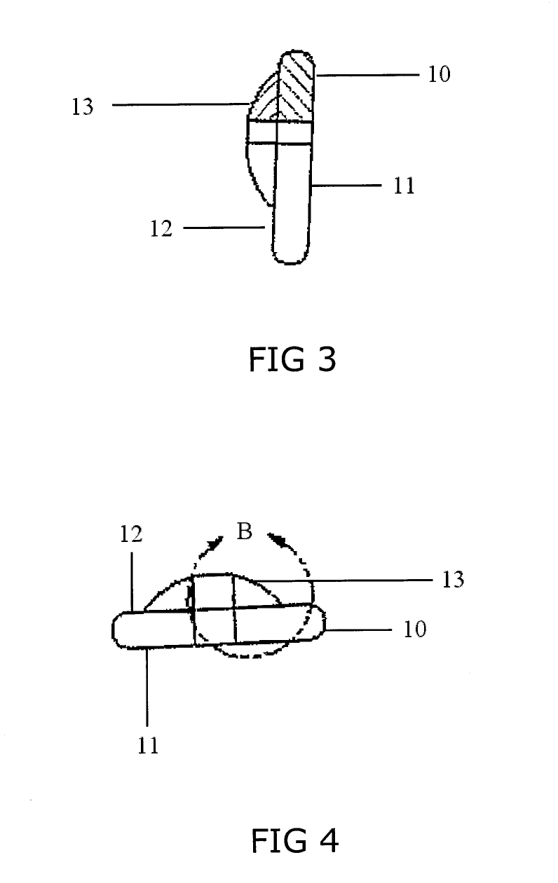

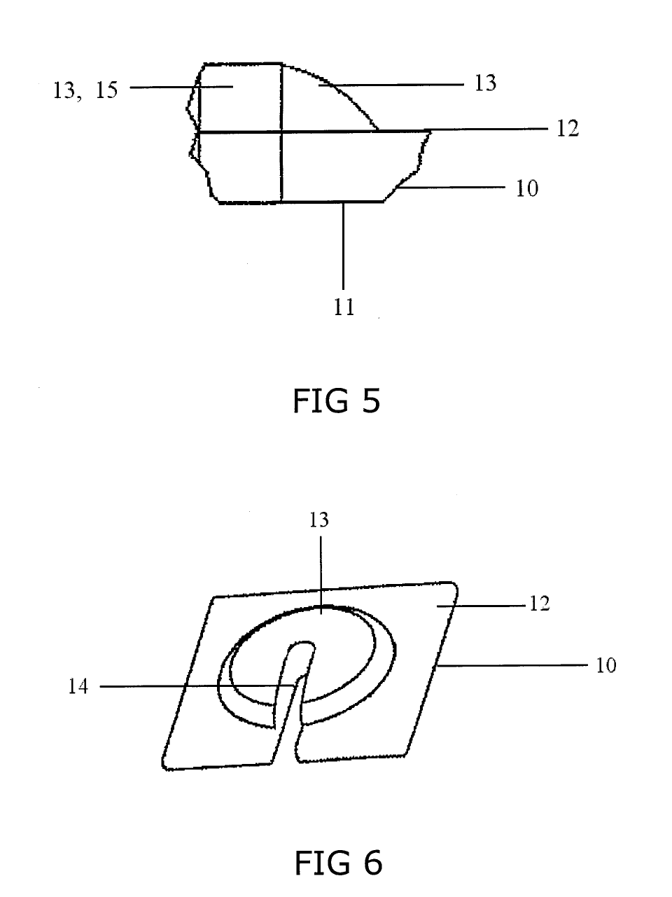

The device of the present invention includes a body 10 having a pressing surface 11 and a contact surface 12. The contact surface 12 further includes a raised portion 13, which in the embodiment shown in FIGS. 1 to 5 is substantially hemispherical. A slot 14, having a diameter sized to accommodate the tube diameter of a supra-pubic catheter, extends from one side edge of the device towards the centre portion of the body 10. In this way, the end 15 of the slot 14 is of maximum depth.

Without offering a definitive explanation of the functioning of the device, it appears that the application of pressure on the pressing face 11 by a user reduces muscle cramping and involuntary muscle spasms experienced by the patient on removal of the catheter and allows an easier and more comfortable removal of the catheter in the patient's body and the subsequent re-insertion of a new catheter. The use of the device of the present invention also reduces tissue trauma and bleeding into the bladder in the hours after the catheter replacement. The net impact of the device is that the patient has less fear and anxiety and provides an improved professional experience for the medical clinician carrying out the procedure.

The body 10 is about 55 mm square and 4 mm thick between surfaces 11 and 12, with the height of the raised portion 14 above the surface 12 being about 5 mm. The overall diameter of the body 10 is about 30 mm. The slot 14 is about 6 mm wide and is adapted to receive the tube diameter of a supra-pubic catheter therein. When the catheter is inserted into a patient's body using the device of the present invention, the tube of the catheter passes through the slot 14 of the body 10 and the slot is adapted to position the tube of the catheter in an orientation that is substantially perpendicular to the patient's abdomen. As described above, the slot 14 extends from a side edge of the device towards a centre portion thereof, such that the end 15 of the slot is located in the middle of the raised portion 13, so that the end of the slot is about 9 mm deep and the catheter is in the centre of the body 10.

The device is manufactured from a surgical grade synthetic polymer and is a sterile, single use “throw-away” item. Alternatively, the device can be manufactured from any other surgical grade material, such as plastic, metal or the like. This assists in reducing the possibility of the device causing the entry point of the catheter in the patient's abdomen to become infected or even resulting a more serious infection in the patient's bladder and/or kidneys. Being a single use “throw-away” item also assists the medical clinician to perform their duties in a prompt, efficient, hygienic and safe manner.

In use, when removing and inserting the supra-pubic catheter in a patient, the body 10 of the device is placed on the body of the patient with the contact surface 12 facing the body and in contact with the patient's skin. As the present invention is particularly suited for use with supra-pubic renal catheters, the contact surface 12 will typically be placed against the abdomen of the patient. The device is positioned around the catheter so that the catheter is abutting the end 15 of the slot 14.

When the catheter is withdrawn or inserted, the medical clinician (or other user of the device) applies pressure by hand to the pressing surface 11, so that the raised portion 13 is pressed into the flesh of the patient around the catheter. This has the effect of widening and realigning the entry point of the catheter in the patient's abdomen, so that sliding contact between the flesh and the catheter is minimized. This reduces the level of pain experienced by the patient when the catheter is inserted or removed. This widening and realignment of the entry point of the catheter into the patient's abdomen also assists the medical clinician in reinserting a new catheter into the patient, as the hole into which the catheter must be inserted is widely opened.

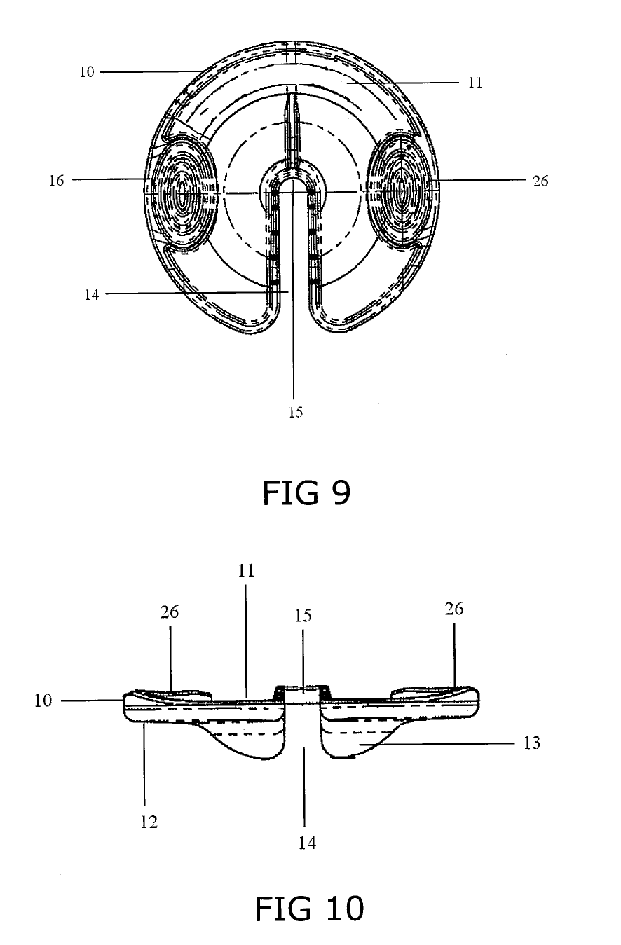

Further embodiments of the present invention are shown in FIGS. 6 to 10. As these further embodiments are similar to the embodiment of the device shown in FIGS. 1 to 5, corresponding features have been labeled with the same nomenclature.

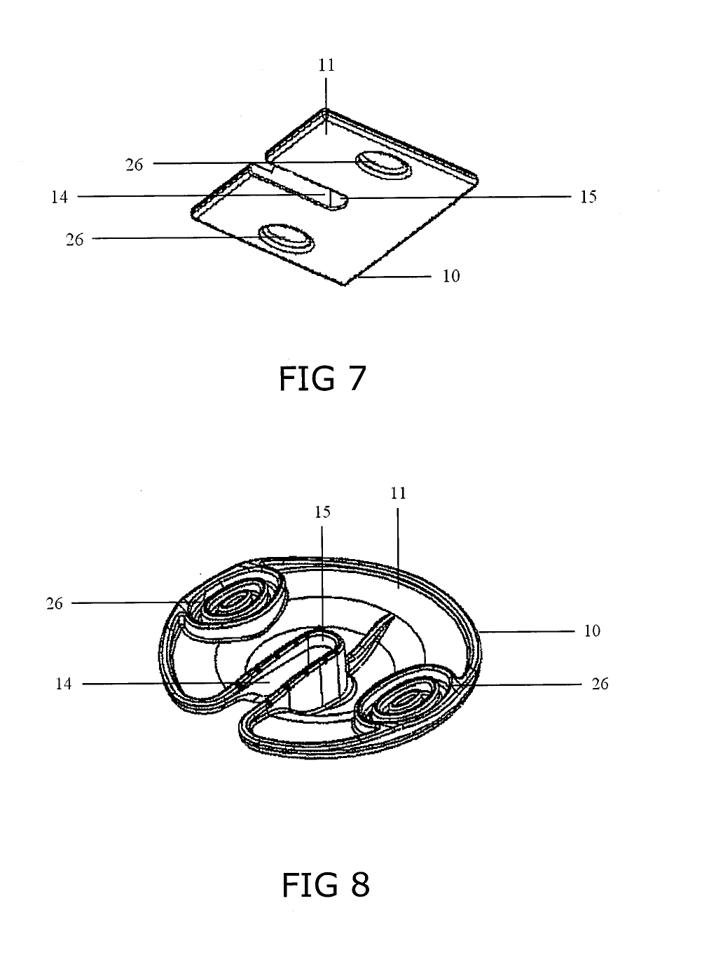

Similar to the embodiment shown in FIGS. 1 to 5, the embodiment of the present invention shown in FIGS. 6 and 7 depicts the body 10 of the device consisting of a pressing surface 11 and a contact surface 12. The contact surface 12 further includes a raised portion 13. However, the raised portion 13 of the embodiment shown in FIGS. 6 and 7 has a substantially flat top surface that is adapted to contact the patient's skin. As best shown in FIG. 7, the pressing surface 11 incorporates two pressure zones in the form of finger depressions 26, to locate the pressure applied by the user on the pressing surface evenly about the catheter entry point in the patient's abdomen.

A third embodiment of the present invention is shown in FIGS. 8 to 10. In this embodiment, the contact surface 12 includes a substantially convex raised portion 13 and the pressure zones 26 on the pressing surface 11 are raised finger locating pads.

It should be understood that any suitable shape may be utilised for the body 10 of the device of the present invention and not just those shapes shown in the accompanying drawings. It should be also understood that in other embodiments not shown in the drawings, the slot 14 may instead be an aperture in the body 10 being of sufficient diameter to fit over at least one end of the catheter.

Unless the context indicates otherwise, the words “comprise” or “comprising” and the like terms should be construed in an inclusive sense, as contrasted to an exclusive or exhaustive sense. These words normally mean “including but not limited to”.

It will be apparent that obvious variations or modifications may be made in accordance with the spirit of the invention that are intended to be part of the invention, and any such obvious variations or modification are therefore within the scope of the invention.

1. A device for the removal and insertion of a supra-pubic catheter in a patient, the device having a body comprising:
a contact surface for contacting with the patient's skin;
an opposed pressing surface adapted to have pressure applied thereon by a user; and
a slot extending from a side edge of the body towards the centre thereof, the slot being adapted to receive an end of said catheter therein,
wherein the contact surface includes a raised portion adapted to compress the patient's skin when said pressure is applied to the pressing surface.

2. A device according to claim 1 wherein, in use, when removing and inserting the supra-pubic catheter in a patient, the raised portion is adjacent to the catheter entry point into the patient's abdomen.

3. A device according to claim 1 wherein the raised portion is at least partially convex.

4. A device according to claim 2 wherein, in use, when removing and inserting the supra-pubic catheter in a patient, the device is placed around the catheter so that the catheter abuts an end portion of the slot at or near the centre of the body.

5. A device according to claim 1 wherein the slot is adapted to receive the tube of the catheter therein, such that when the catheter is inserted in a patient, the tube of the catheter is substantially perpendicular to the patient's abdomen.

6. A device according to claim 2 wherein, in use, when removing and inserting the supra-pubic catheter in a patient, a user applies the pressure to the pressing face and the raised portion depresses the region of the patient's abdomen around the entry point of the catheter thereby widening and realigning the entry point of the catheter in the patient's abdomen.

7. A device according to claim 5 wherein the widening and realigning of the entry of the catheter in the patient's abdomen eases the pain and tissue trauma experienced during removal and insertion of the catheter.

8. A device according to claim 1 wherein the pressing surface includes two pressure zones, which are adapted to have said pressure applied thereon, the pressure zones locate the pressure application evenly about the entry point of the catheter in the patient's abdomen.

9. A device according to claim 8 wherein said pressure zones are marked with distinctive indicia to indicate to the user where to apply the pressure on the pressing surface.

10. A device according to claim 8 wherein said pressure zones are finger depressions.

11. A device according to claim 8 wherein said pressure zones are raised finger locating pads.

12. A device according to claim 1 manufactured from surgical grade material.

13. A device according to claim 12 wherein said material is surgical grade synthetic polymer.

14. A device according to claim 1 being a sterile, single use “throw-away” item.

15. A method of removing and inserting a supra-pubic catheter in a patient having the steps of:
locating a device adjacent to the entry point of the catheter in the patients abdomen to fit around the catheter;
applying pressure onto the device to depress the region of the patient's abdomen around the entry point of the catheter thereby widening and realigning the entry point of the catheter in the patient's abdomen;
whilst the pressure is being continually applied onto the device, removing the catheter from the patient and subsequently inserting a new catheter into the patient,
wherein the device has a body comprising a contact surface for contacting with the patient's skin; an opposed pressing surface adapted to have pressure applied thereon by a user; and a slot extending from a side edge of the body towards the centre thereof, the slot being adapted to receive an end of said catheter therein, and wherein the contact surface includes a raised portion adapted to compress the patient's skin when said pressure is applied to the pressing surface.

16. A method of removing and inserting a supra-pubic catheter according to claim 15 wherein the widening and realigning of the entry of the catheter in the patient's abdomen eases the pain and tissue trauma experienced during removal and insertion of the catheter.

17. A method of removing and inserting a supra-pubic catheter according to claim 15 wherein the slot is adapted to receive the tube of the catheter therein, such that when the catheter is inserted in a patient, the tube of the catheter is substantially perpendicular to the patient's abdomen.

18. A method of removing and inserting a supra-pubic catheter according to claim 15 wherein, in use, when removing and inserting the supra-pubic catheter in a patient, the raised portion is adjacent to the catheter entry point into the patient's abdomen.

19. A method of removing and inserting a supra-pubic catheter according to claim 16 wherein, in use, when removing and inserting the supra-pubic catheter in a patient, the device is placed around the catheter so that the catheter abuts an end portion of the slot at or near the centre of the body.

20. A method of removing and inserting a supra-pubic catheter according to claim 15 wherein the pressing surface includes two pressure zones, which are adapted to have said pressure applied thereon, the pressure zones locate the pressure application evenly about the entry point of the catheter in the patient's abdomen.
